(19) United States
(12) Patent Application Publication
(10) Pub. No.: US 2012/0040004 A1
(43) Pub. Date: Feb. 16, 2012

(54) TREATMENT OF RADIATION-INDUCED FIBROSIS

(75) Inventors: Kenneth Alan Howard, Arhus N (DK); Jørgen Kjems, Risskov (DK); Isabel Nawroth, Arhus C (DK); Jan Alsner, Hojbjerg (DK); Jens Overgaard, Arhus C (DK); Flemming Besenbacher, Arhus V (DK)

(73) Assignees: REGION MIDTJYLLAND, Viborg (DK); AARHUS UNIVERSITET, Arhus C (DK)

(21) Appl. No.: 13/146,498

(22) PCT Filed: Jan. 28, 2010
(86) PCT No.: PCT/DK2010/050021

§ 371 Date: Oct. 27, 2011

(30) Foreign Application Priority Data
Jan. 28, 2009 (DK) PA 2009 00132

(51) Int. Cl. A61K 31/713; A61P 43/00; A61K 9/14; B82Y 5/00

(52) U.S. Cl. 424/489; 514/44 A; 977/773; 977/906

(57) ABSTRACT

The present invention relates to treatment or prevention of radiation induced fibrosis using TNF-alpha antagonism. Preferably, TNF-alpha is antagonized by direct binding or by inhibition of synthesis. In a preferred embodiment, the invention comprises intraperitoneal administration of a chitosan-siRNA nanoparticle, wherein the siRNA is targeted to the mRNA encoding TNF-alpha.

### TECHNICAL FIELD OF THE INVENTION

The present invention relates to the treatment of radiation-induced fibrosis (RIF), which is one of the most adverse long-term effects of radiation-based cancer therapy. More specifically, the present invention relates to TNF-alpha antagonism for treatment of RIF. A preferred method of TNF-alpha antagonism uses chitosan/TNF-alpha-specific siRNA nanoparticles introduced by an intraperitoneal route for downregulation of TNF-alpha and it has surprisingly been demonstrated that this approach very effectively prevents the development of RIF.

### BACKGROUND OF THE INVENTION

### TNF-α

Tumor necrosis factor (TNF, TNF-a, TNF-α, cachexin or cachectin and formally known as tumor necrosis factor-alpha) is a cytokine involved in systemic inflammation and is a member of a group of cytokines that stimulate the acute phase reaction. The primary role of TNF is in the regulation of immune cells. TNF-α is also able to induce apoptotic cell death, to induce inflammation, and to inhibit tumorigenesis and viral replication.

The role of TNF-α in fibrosis is controversial as Distler et al. has discussed recently. Distler et al. summarize that results from in vitro and in vivo studies are contradictory and do not allow definite conclusions about the role of TNF-α in fibrosis. On one hand the majority of in vitro studies show antifibrotic effects of TNF-alpha, in that it suppresses the production of collagen, reduces the expression of TIMPs (tissue inhibitor of metalloproteinase 1), and stimulates the release of MMPs (matrix metalloproteinases), thereby preventing the accumulation of ECM (extracellular matrix). On the other hand, a number of in vivo studies suggest a profibrotic role of TNF-alpha. The authors speculate that the difference between in vitro and in vivo studies might be explained by the inflammatory component in animal models of experimental fibrosis. (i.e. TNF-alpha may act profibrotic in animal models of experimental fibrosis because of its general pro-inflammatory effects.) Therefore to predict the effects of anti-TNF-alpha treatments in human fibrotic diseases, the key question is whether inflammation triggers and perpetuates the development of fibrosis in humans. The authors state that the role of inflammation in human fibrotic diseases is less clear than was initially thought and might differ between the specific organs and might also depend on other parameters such as disease duration.

Thus, TNF-α is a major proinflammatory stimulus and activates inflammatory cells but the role of inflammation in human fibrotic diseases seems unclear.

In another chronic inflammatory disease, arthritis, intervention of the inflammatory cascade in mice with anti-TNF-α monoclonal antibodies provides the basis for clinical immunotherapy treatments although cost and possible auto-immunity to antibodies might be a problem. Based on this knowledge, Howard et al. demonstrate in their study nanoparticle-mediated TNF-α knockdown in peritoneal macrophages as a novel strategy for arthritis treatment.

Howard et al. suggest that their work and that of others using liposomal carriers promote the i.p. route for induction of TNF-alpha-siRNA-mediated systemic anti-inflammatory effects. In line with the disease model employed, only rheumatoid arthritis is suggested as a specific condition that may be treated by i.p administration of the described nanoparticles.

Thus, proving a role of TNF-α in arthritis and finding a treatment seems to be closer than ever, but in other more distantly related fibrotic diseases like radiation-induced fibrosis (RIF) remains, until now, an enigma.

### Radiation Induced Fibrosis

One of the most common long-term adverse effects of radiation-based cancer therapy is radiation-induced fibrosis (RIF) as a late effect of radiation therapy (RT) in skin and subcutaneous tissue, lungs, the gastrointestinal (GI) and genitourinary (GU) tracts, muscles, or other organs, depending upon the treatment site. RIF may cause both cosmetic and functional impairment, which can lead to death or a significant deterioration in the quality of life.

The development of RIF is influenced by multiple factors, including the radiation dose and volume, fractionation schedule, previous or concurrent treatments, genetic susceptibility, and co-morbidities such as diabetes mellitus. Although RIF originally was assumed to be a slow, irreversible process, contemporary studies suggest that RIF is not necessarily a fixed process.

RIF is a complex biological process developing gradually over a number of years that restricts the dose of radiation. RIF is believed to occur as the result of a coordinated response to radiation involving several different cytokines and growth factors, fibroblast proliferation and differentiation, and also remodelling of the extracellular matrix. The process which leads to RIF is very complex and includes Smad 3, CTGF (Connective Tissue Growth Factor), the Rho/ROCK pathway and pro-inflammatory cytokines such as IL-1, IL-8 and IFN-γ.

Immediately after irradiation, initiation of cytokine production is induced and continues as a cascade during the whole process of cutaneous radiation syndrome which will lead to progressive late symptoms, mainly fibrosis. Major cytokines which are released by skin cells include IL-1, IL-6, TNF-α and TGF-β.

Due to this complex cytokine network, the role of cytokines in radiation-induced fibrosis is only beginning to be elucidated.

It is unclear whether a beneficial therapeutic effect in relation to RIF can be achieved by modulating the activity of any one of the cytokines or combinations thereof.

The prevention of RIF has focused on improvements in RT technique, which have resulted in higher doses to the tumour target and decreased doses to normal tissue, thus potentially preventing the development of RIF. Furthermore, established RIF may be treatable with novel therapeutic approaches. A variety of strategies have been tested, including the combination of pentoxifylline (PTX) and vitamin E (alpha-tocopherol). However, reports on PTX appear contradictory possibly due to degradation in systemic administration.

Another agent is SOD (Cu/Zn superoxide dismutase) but it is problematic because of its short biological half-life, relatively high molecular weight (33 kDa) and hydrophilic nature.

WO06/020230 describes a method of treating fibrotic disease comprising administration of siRNAs. The patent application mentions i.p. administration as one of many possible administration routes. It is described that the siRNA can be embedded within or associated with a delivery matrix. Among many potential forms of the matrix, a nanoparticle is mentioned and among many different materials for the matrix, chitosan is mentioned. The siRNA described in the application interferes with a PLOD2 gene and inhibit translation of a TLH protein or a protein involved in the production or processing of a TLH protein. Thus, the method of this patent application seeks to reduce or prevent aberrant crosslinking of collagen and the consequent formation of fibrotic tissue.

Other than the approaches mentioned (SOD, PTX alone, or with tocopherol, and siRNAs against crosslinking of collagen), there have been no other significant clinical data showing significant interference with the RIF process.

Hence, an improved treatment of RIF would be advantageous, and in particular a more efficient and/or reliable target and administration would be advantageous.

### SUMMARY OF THE INVENTION

Based on experimental validation of TNF-alpha as a target for treatment of RIF, the present invention provides TNF-alpha antagonists for use in the treatment or prevention of radiation induced fibrosis. In the broadest aspect, the TNF-alpha antagonist may be any compound capable of antagonising the activity of TNF-alpha.

In a preferred embodiment, the TNF-alpha antagonist binds directly to TNF-alpha. A direct binder may be an antibody, an aptamer or a soluble receptor. A number of such antagonists are well known to the skilled man and some of them are marketed for treatment of inflammatory diseases. These include Infliximab (Remicade), Adalimumab (Humira) and Etanercept (Enbrel).

In another embodiment, the TNF-alpha antagonist inhibits synthesis of TNF-alpha. In this embodiment, the antagonist may be a siRNA directed to the TNF-alpha mRNA or an RNase H activitating antisense molecule directed to the TNF-alpha mRNA. As the skilled man will recognize such oligonucleotides mediates degradation of their target mRNA and thereby decreases synthesis of the protein encoded by the mRNA. When the TNF-alpha antagonist is an oligonucleotide, it is preferred that the oligonucleotide is formulated in a liposome or a nanoparticle. A preferred nanoparticle is a chitosan nanoparticle.

Preferably, the nanoparticle is a chitosan-siRNA nanoparticle, wherein the siRNA is targeted to the mRNA encoding TNF-alpha.

Preferably, treatment comprises intraperitoneal administration of the TNF-alpha antagonist. In addition to alleviating effects of radiation therapy, the use of the invention enables radiation therapy at higher doses or at shorter intervals.

### BRIEF DESCRIPTION OF THE FIGURES

FIG. 1: Characteristic progress of the acute phase in radiation-induced fibrosis and chitosan/siRNA nanoparticles treatment strategy.

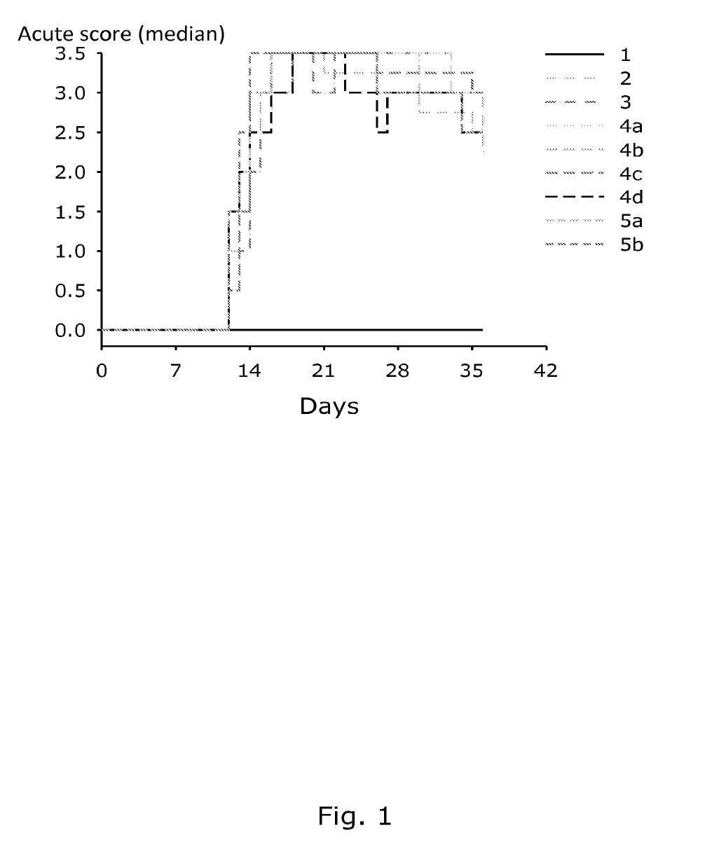

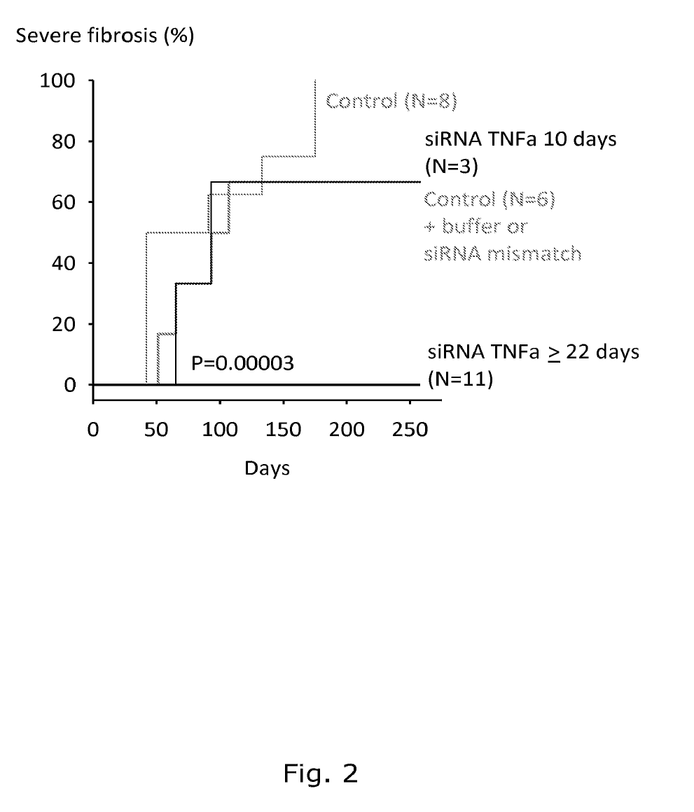

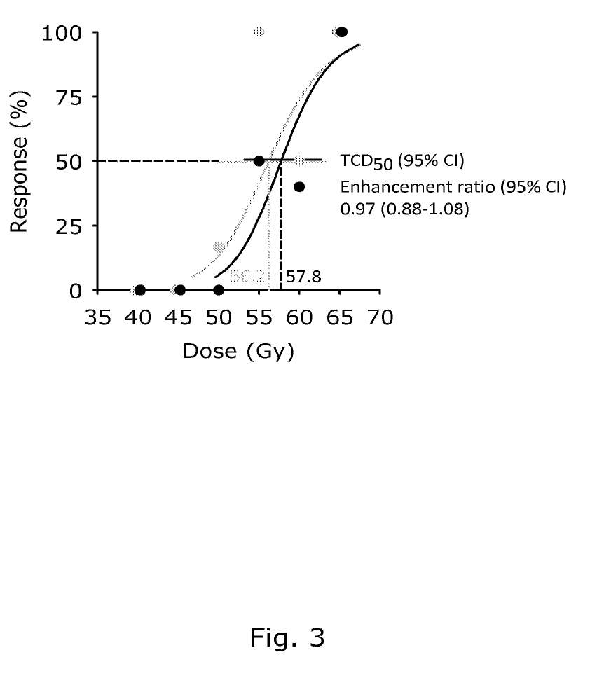

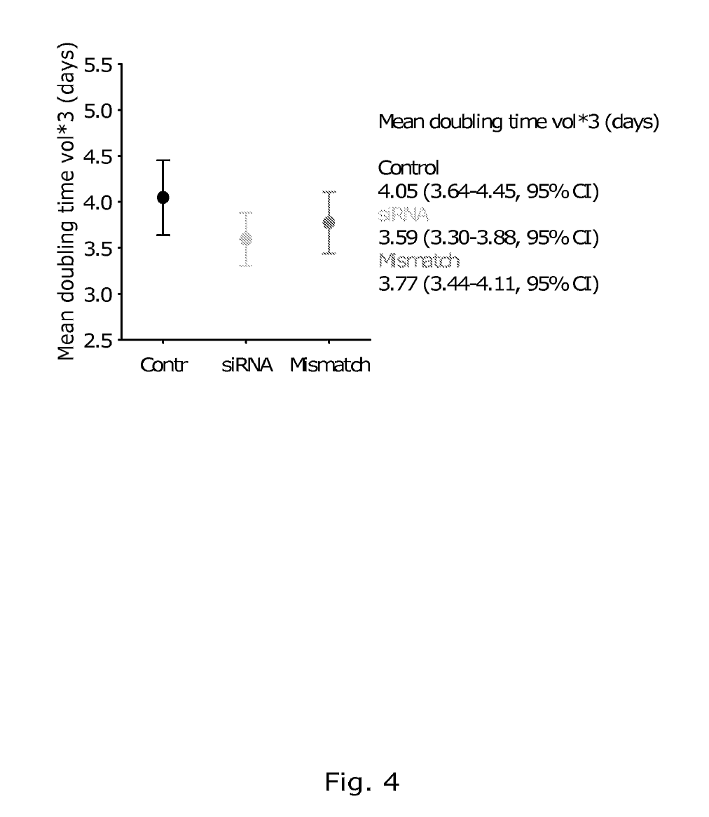

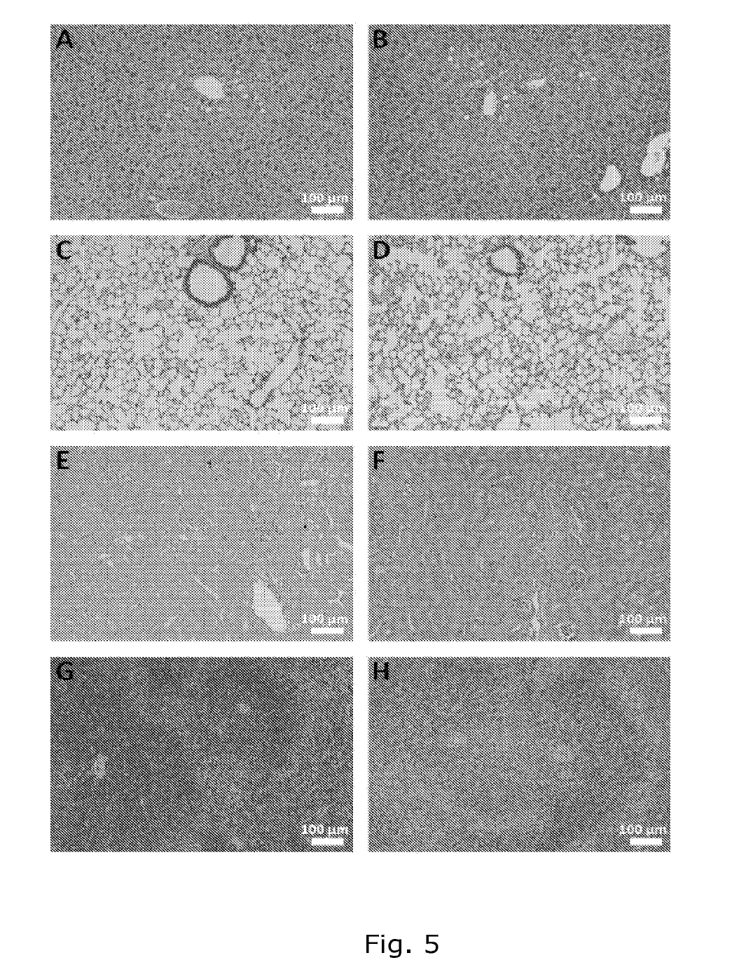

Radiation-induced fibrosis occurs as the result of a coordinated response to irradiation (IR) involving several different cytokines (e.g. TNF-α) and growth factors which levels are elevated during the acute phase. At day 12 after a radiation dose of 45 Gy the acute phase arises and endures until day 42.

The acute phase is phenotypic characterized and scored by inflamed skin of the irradiated hind leg. Group 1: control, no IR, no treatment (n=3); Group 2: buffer, 2d before IR (n=3); Group 3: siRNA mismatch, 2d before IR (n=3); Group 4a: TNF-α siRNA, 2d before IR until day 10 (n=3); Group 4b: TNF-α siRNA, 2d before IR until day 22 (n=3); Group 4c: TNF-α siRNA, 2d before IR until day 34 (n=3); Group 4d: TNF-α siRNA, 2d before IR until day 225 (n=3); Group 5a: TNF-α siRNA, 1d after IR until day 22 (n=3); Group 5b: TNF-α siRNA, 1d after IR until day 225 (n=3).

FIG. 2: Prevention of fibrotic condition in CDF1 mice after i.p. administration of chitosan/siRNA nanoparticles.

The irradiated hind leg of the mice was scored for clinical symptoms of radiation-induced fibrosis by using the leg contracture model for level of severe fibrosis (scale: 0=normal, 1-2=mild to moderate fibrosis, 3-4=severe fibrosis). Animals were dosed i.p. with 200 μl chitosan nanoparticles containing 5 μg TNF-α siRNA (either 2 days before or 1 day after irradiation, black lines) and either 5 μg negative control or sodium acetate buffer (2 days before irradiation, grey thick line). A group of untreated animals is included (grey thin line). The chitosan/siRNA nanoparticles treatment was continued twice a week and terminated on day 10 (black thin line), and days 22, 34 or 258 (black thick line). Treatment with negative control or buffer was terminated on day 22.

The development of severe fibrosis is significantly higher in the combined control groups (N=14) compared to the group of animals treated with TNF-α siRNA for 22 or more days (P=0.00003). Data from single experiment.

FIG. 3: Effect on Chitosan/siRNA nanoparticles treatment on tumour control.

Male CDF1 mice were injected with tumour cells and i.p. dosed with 200 μl chitosan/siRNA nanoparticles (grey line) as the tumour achieved a size of 200 mm3. 2 days after the first i.p. injection irradiation was given in different dose (40 Gy, 45 Gy, 50 Gy, 55 Gy, 60 Gy and 65 Gy) and chitosan/siRNA treatment was continued twice a week for 3 month. A control group (black line) is included.

FIG. 4: Effect on Chitosan/siRNA nanoparticles treatment on tumour growth.

Animals were dosed i.p. with 200 μl chitosan nanoparticles containing 5 μg TNF-α siRNA (light grey stock) or 5 μg negative control (grey stock). A group of untreated animals is included (black stock).

FIG. 5: Histopathological evaluation of liver, lung, kidney and spleen from CDF1 mice dosed i.p. with chitosan/DsiRNA nanoparticles. Histological sections from the organs were taken after the end of the study (day 258). A: control, liver. B: TNFα DsiRNA, liver. C: control, lung. D: TNFα DsiRNA, lung. E: control, kidney. F: TNFα DsiRNA, kidney. G: control, spleen. H: TNFα DsiRNA, spleen. Representative images of the groups are shown (magnification: objective 10×).

The present invention will now be described in more detail in the following.

### DETAILED DESCRIPTION OF THE INVENTION

It should be noted that embodiments and features described in the context of one of the aspects of the present invention also apply to the other aspects of the invention.

All patent and non-patent references cited in the present application, are hereby incorporated by reference in their entirety.

TNF-a, TNF-α, cachexin or cachectin as used herein refers to the cytokine formally known as tumor necrosis factor-alpha, preferably of human origin.

As documented in the examples section, the inventors have been able to deliver the siRNA of a chitosan/siRNA nanoparticle to peritoneal macrophages and downregulate the target mRNA of the siRNA. Surprisingly, the inventors have discovered that this method can be used to treat radiation induced fibrosis (RIF), when the siRNA is targeted to TNF-alpha. Not intended to be bound by theory, the results indicate that TNF-alpha plays a pivotal role in the development of RIF. Therefore any TNF-alpha antagonists may be used for treatment or prevention of RIF. Moreover, a particular favourable route of administration of TNF-alpha antagonists seems to be intraperitoneal administration, perhaps because of downregulation of TNF-alpha production in peritoneal macrophages. Regardless of the mechanisms involved (be they understood or not), the present inventors have demonstrated that intraperitoneal administration of siRNA directed to TNF-alpha is surprisingly effective in treating RIF. Moreover, it was observed that TNF-alpha inhibition did not seem to have an unwanted effect on tumour growth, as might be expected since TNF-alpha is known to inhibit tumorigenesis (example 2).

No similar treatment for radiation-induced fibrosis has been reported or published before. There are many cytokines involved in fibrosis besides TNF-alpha and it is surprising that antagonism of just one of the cytokines proves effective for prevention of RIF. Small amounts of siRNA gave very convincing results—the positive effect of the chitosan/siRNA treatment was lasting until the end of the study and the mice responded well to the treatment although i.p. dosed twice a week for 258 days (example 1).

Treatment with the TNF-alpha antagonists of the present invention may enable a higher dose of radiation which will make the tumour radiation treatment more efficient (to kill tumour cells in the most efficient way, higher doses of irradiation are necessary). Moreover, when treatment is done via i.p. injections patient compliance will be enhanced and there should be fewer problems concerning administration (e.g. in diabetes with insulin i.p. injections).

To evaluate any possible systemic cytotoxic effect of long-term administration of chitosan/DsiRNA nanoparticle, mice that had been continuously bi-weekly treated were sacrificed after 258 days (total 78 i.p. injections) and liver, lung, spleen and kidney taken for histopathological analysis. No significant histological abnormalities were observed in the analysed organs between non-treated and TNFα DsiRNA treated animals (example 3).

A first aspect of the invention is a TNF-alpha antagonist for use in the treatment or prevention of radiation induced fibrosis. As used in the following, the term “treatment” covers both treatment and prevention.

In its broadest aspect, the TNF-alpha antagonist may be any compound capable of antagonising the activity of TNF-alpha. More preferably, the TNF-alpha antagonists interact with either the TNF-alpha protein or the mRNA encoding TNF-alpha.

An embodiment of the present invention relates to an siRNA targeting TNF-alpha for use in the treatment or prevention of radiation induced fibrosis.

In another embodiment of the present invention is the siRNA formulated in a liposome or a nanoparticle. A preferred nanoparticle is a chitosan nanoparticle.

In a preferred embodiment, the TNF-alpha antagonist binds directly to TNF-alpha. A direct binder may be an antibody, an aptamer or a soluble receptor. A number of such antagonists are well known to the skilled man and some of them are already marketed for treatment of inflammatory diseases. These include Infliximab (Remicade), Adalimumab (Humira) and Etanercept (Enbrel).

In another embodiment, the TNF-alpha antagonist interacts with the mRNA encoding TNF-alpha to thereby inhibit synthesis of TNF-alpha. In this embodiment, the antagonist may be a siRNA directed to the TNF-alpha mRNA or an RNase H activitating antisense molecule directed to the TNF-alpha mRNA. As the skilled man will recognize such oligonucleotides mediate degradation of their target.

When the TNF-alpha antagonist is an oligonucleotide, it is preferred that the oligonucleotide is formulated in a liposome or a nanoparticle. A preferred nanoparticle is a chitosan nanoparticle.

The term siRNA as used herein is a RNA complex that recruits the so-called RNA-Induced-Silencing-Complex (RISC) and mediates translational repression or degradation of target mRNAs. Alternatives to the term siRNA are Dicer-substrate siRNA (DsiRNA) or microRNA (miRNA). Preferred is degradation of target mRNAs. The skilled artisan will recognize that different siRNA structures exist and that they may be used interchangeably. Typically, the siRNAs are double stranded RNAs of 20-23 nt with 3′overhangs. However, the siRNAs may be longer, e.g. 27 nt or longer. Such longer siRNAs have been referred to as dicer substrates or Dicer-substrate siRNA (DsiRNA). The siRNAs may also be blunt ended, have 5′overhangs and they may be chemically modified, e.g. with LNAs or 2′O-methyls.

As mentioned, RNase H inducing antisense oligonucleotides may be used as alternatives to siRNAs since these can be targeted to the same mRNAs and also mediate degradation. Thus, the oligonucleotide may be a siRNA or an RNase H inducing antisense oligonucleotide targeted to TNF-alpha and it should be clear that whenever reference is to a siRNA, RNase H inducing antisense oligonucleotides or Dicer-substrate siRNA (DsiRNA) may be used instead. Ribozymes targeted to the mRNA encoding TNF-alpha may also be used. However, siRNAs or DsiRNA are preferred over RNase H inducing antisense oligonucleotides and ribozymes.

An embodiment of the present invention relates to a TNF-alpha antagonist of the present invention, wherein the antagonist interacts with the mRNA encoding TNF-alpha or binds directly to TNF-alpha.

An embodiment of the present invention relates to a TNF-alpha antagonist of the present invention, wherein the antagonist is selected from the group consisting of an siRNA directed to the mRNA encoding TNF-alpha, an antibody, an aptamer, a soluble TNF-alpha receptor that binds directly to TNF-alpha, or an RNase H inducing antisense oligonucleotide directed to the mRNA encoding TNF-alpha, and a ribozyme directed to the mRNA encoding TNF-alpha.

In an embodiment of the present invention is the TNF-alpha antagonist of the present invention an siRNA.

In another embodiment of the present invention is the TNF-alpha antagonist of the present invention formulated in a chitosan/siRNA nanoparticle.

In another embodiment of the present invention is the TNF-alpha antagonist of the present invention formulated for intraperitoneal administration.

In another embodiment of the present invention is the TNF-alpha antagonist of the present invention formulated for administration prior to initiation of radiation therapy.

In another embodiment of the present invention administration is of the TNF-alpha antagonist of the present invention initiated least 24 hours before radiation therapy.

In another embodiment of the present invention is the administration of the TNF-alpha antagonist of the present invention initiated no more than 1 week before radiation therapy.

In another embodiment of the present invention is the administration of the TNF-alpha antagonist of the present invention done in repeating cycles of administration and radiation therapy.

In another embodiment of the present invention is the time between radiation therapies reduced in comparison with the same radiation therapy without administration of TNF-alpha antagonist formulation of the present invention.

In another embodiment of the present invention is the radiation dose increased as compared to radiation therapy without administration of TNF-alpha antagonist formulation of the present invention.

An embodiment of the present invention relates to a method of treating radiation induced fibrosis comprising administering an therapeutically effective amount of a TNF-alpha antagonist of the present invention to a person in need thereof. Administration of the TNF-alpha antagonists can be by the systemic route (e.g. intravenous or intraperitoneal) or mucosal route (e.g. pulmonary or oral). Preferably, treatment comprises intraperitoneal (i.p.) administration/injection.

The treatment is initiated with a first injection and is usually followed by subsequent injections.

In a preferred embodiment, treatment is initiated before radiation therapy.

Treatment may e.g. be initiated at least 24 hours before radiation therapy.

In another embodiment, treatment is initiated no more than 1 week before radiation therapy such as 7 days before radiation, such as 6 days before radiation, such as 5 days before radiation, such as 4 days before radiation, such as 3 days before radiation, such as 2 days before, such as 1 day before.

In another embodiment is the treatment initiated less than 24 hours before radiation therapy.

In yet another preferred embodiment, repeating cycles of treatment and/or radiation therapy is performed such as at least once a week, twice a week, three times a week, four times a week, five times a week, 6 times a week or every day.

Treatment with the TNF-alpha antagonist may allow a shorter interval between radiation doses. Thus, in one embodiment repeating cycles of treatment and radiation therapy is performed, wherein the time between radiation therapy is reduced in comparison with the same radiation therapy without nanoparticle treatment.

Preferably, the interval of TNF-alpha antagonist treatment is twice a week.

In another embodiment, treatment is continued with i.p. injections of TNF-alpha antagonist twice a week for at least 100 days after the last radiation therapy.

More preferably, treatment is continued for at least 200 days, 250 days, at least 300 days or at least 400 days. Instead of i.p. injections of TNF-alpha antagonist twice a week, IP injections may be given every week, every second week or once a month.

Treatment with the TNF-alpha antagonist may enable a higher dose of radiation, i.e. the TNF-alpha antagonist may be used to increase the acceptable dose of radiation. Therefore, in one embodiment, the radiation dose is increased as compared to radiation therapy without TNF-alpha antagonist administration. Preferably, the dose is increased at least 10%, 20%, 30%, 40% or 50%.

As will be clear, the person to be treated with the TNF-alpha antagonist is undergoing radiation therapy. Typically, the person has a cancer selected from the group consisting of head- and neck cancer and breast cancer but any types of cancer whose treatment promote RIF are relevant. If patients with these kind of cancers receive radiotherapy, RIF is one of the most common long-term adverse effects and is therefore a dose limiting factor.

Preferably, the dose of the TNF-alpha antagonist is selected from the group consisting of less than 50 mg/kg/day, less than 40 mg/kg/day, less than 30 mg/kg/day, less than 20 mg/kg/day, less than 10 mg/kg/day, less than 5 mg/kg/day, less than 1 mg/kg/day and less than 1 mg/kg/day.

In another preferred embodiment, the dose is selected from the group consisting of less than 50 mg/kg/week, less than 40 mg/kg/week, less than 30 mg/kg/week, less than 20 mg/kg/week, less than 10 mg/kg/week, less than 5 mg/kg/week and less than 1 mg/kg/week.

When referring to dose, reference is to the amount of bioactive agent (e.g. siRNA, RNase H inducing antisense oligonucleotide, antibody, and soluble receptor) in the dose. In another embodiment, reference is to the amount of material (e.g. lipid formulation or nanoparticle) in the dose.

### Preparation of Nanoparticle

A particular preferred TNF-alpha antagonist is a chitosan/siRNA nanoparticle.

In the present context refers chitosan/siRNA nanoparticle to a nanoparticle that comprises both chitosan and at least one siRNA.

Preferably, this nanoparticle for use in treatment of radiation induced fibrosis is prepared by a method comprising
a. Providing a chitosan solution
b. Providing an siRNA solution
c. Mixing the solution of step a with the solution of step b
d. Incubating the solution of step c under conditions of complex formation such that chitosan/siRNA nanoparticles form.

In one embodiment, the chitosan/siRNA nanoparticle comprises an initial crosslinker, such as polyphosphate.

In another preferred embodiment of the method of preparing a chitosan/RNA nanoparticle, the chitosan does not comprise an initial crosslinker.

The term initial cross linker is used to for a crosslinker that is added to chitosan to form a particle, before the RNA molecule is added. Typically in the art, e.g. when chitosan/plasmid nanoparticles are formed, the particles are preformed using an initial crosslinker such as polyphosphate. Thus, it is believed that the structure and activity of a particle formed without an initial crosslinker differ from that of a particle formed with an initial crosslinker. In particular, the use of an initial crosslinker seems to imply that the RNA will be distributed at the surface of the preformed particle, whereas when using the RNA as crosslinker, the RNA will be distributed evenly through the particle. An even distribution is expected to a positive effect on the biostability of the RNA molecules of the nanoparticle, as they will be less accessible for RNases.

Moreover, the omission of the initial crosslinker provides are more facile method of preparation. Instead of a two-step method, where the particles are formed first and then the RNA is added, a one-step method is provided in which the RNA and chitosan is mixed to form nanoparticles directly.

Thus, in a preferred embodiment, the RNA functions as a crosslinker in the formation of a nanoparticle. In other words, the RNA is the formactive component.

### Concentrations

### RNA Concentration

In one of embodiment of the method of preparing a chitosan/RNA nanoparticle, the RNA solution comprises RNA at a concentration selected from the group consisting of at least 5 μM, at least 10 μM, at least 20 μM, at least 30 μM, at least 40 μM, at least 50 μM, at least 60 μM, at least 70 μM, at least 80 μM, at least 90 μM and at least 100 μM and at least 250 μM.

### Chitosan Concentration

In another embodiment, the chitosan solution comprises chitosan at a concentration from the group consisting of at least 50 μg/ml, at least 60 μg/ml, at least 70 μg/ml, at least 80 μg/ml, at least 90 μg/ml, at least 100 μg/ml, at least 110 μg/ml, at least 120 μg/ml, at least 130 μg/ml, at least 140 μg/ml, at least 150 μg/ml, at least 160 μg/ml, at least 170 μg/ml, at least 180 μg/ml, at least 190 μg/ml, at least 200 μg/ml, at least 250 μg/ml, at least 500 μg/ml, at least 750 μg/ml and at least 1000 μg/ml.

### Degree of Deacetylation

Preferably, the chitosan has a relatively high degree of deacetylation. Thus, in one embodiment, the chitosan has a degree of deacetylation of selected from the group consisting of at least 60%, least 65%, least 70%, least 75%, least 80%, least 85% and at least 95%.

### Molecular Weight of Chitosan

The molecular weight of the chitosan is preferably more than 10 kDa. In another embodiment, the molecular weight is more than 50 kDa and even more preferred is a molecular weight of more than 100 kDa.

Chitosan samples with a molecular weight in the range of 100-170 kDa and a deacetylation degree are particular favourable.

### N:P Ratio

The above mentioned parameters can all be used to control the characteristics of the formed nanoparticle. Another important parameter is the so-called N:P ratio, defined herein as the ratio of chitosan amino groups (N) to RNA phosphate groups (P).

In a preferred embodiment, the nanoparticle is formed at a N:P ratio larger than 25. Experiments document that increasing the N:P ratio, leads to larger particles. In other embodiments, the N:P ratio is selected from the group consisting of a N:P ratio larger than 60, larger than 70, larger than 80, larger than 90, larger than 100 and larger than 150.

In this preferred embodiment, wherein the N:P ratio is larger than 70, the RNA solution comprises RNA at a concentration lower than 250 μM, such as lower than 100 μM, such as lower than 90 μM, lower than 80 μM, lower than 70 μM, lower than 60 μM, lower than 50 μM, lower than 40 μM, lower than 30 μM, lower than 20 μM, lower than 10 μM, lower than 5 μM or lower than 1 μM.

When employing a high N:P ratio and a low RNA concentration, nanoparticles can be formed that comprises loosely bound chitosan.

As the degree of loosely bound chitosan is dependent on both the concentration of RNA in the RNA solution and on the N:P ratio, the skilled worker will appreciate how to manipulate these parameters to create nanoparticles with loosely bound chitosan. E.g. a high RNA concentration of the RNA solution may be used, if also the N:P ratio is kept high, i.e. a high concentration of chitosan is used.

In one embodiment, the nanoparticle comprising loosely bound chitosan has a high N:P ratio.

A nanoparticle with loosely bound chitosan is of interest e.g. to improve mucosal delivery. Therefore, in one embodiment, the nanoparticle with loosely bound chitosan is for mucosal delivery.

A nanoparticle particle with a discrete character is of interest e.g. for systemic delivery. Such a particle can also be formed by controlling various parameters involved in the method of forming the nanoparticle. Particularly, a low N:P ratio favours the formation of a discrete nanoparticle. As mentioned above, the concentration of RNA and chitosan can be varied while maintaining a reasonably constant N:P ratio.

Concentrated Method Vs. N:P Ratio

In this embodiment, the N:P ratio is lower than 70 such as but not limited to a N:P ratio lower than 60, lower than 50, lower than 40, lower than 30, lower than 20 or lower than 10, respectively.

In one embodiment, the nanoparticle of discrete character has a low N:P ratio. In another embodiment, the concentration of the RNA solution is at least 100 μM, such as but no limited to at least 250 μM, at least 200 μM, at least 150 μM, at least 90 μM, at least 80 μM, at least 70 μM, at least 60 μM or at least 50 μM, respectively.

Using a high concentration of RNA in the RNA solution turns out to have several advantages. As outlined in the examples section, when the particles are formed using a RNA solution with a concentration of 250 μM, the particles are more discrete and monodispersed, as compared to particles formed using a pre-diluted RNA solution of 20 μM. Moreover, it surprisingly turns out that the nanoparticles formed using the concentrated RNA solution have a more specific effect, i.e. they do not give rise to any non-specific knockdown, which may be the case for particles formed using a RNA solution with a lower concentration of RNA.

Furthermore, using a high concentration of RNA in the RNA solution allows particle formation at a low pH such as ph 4.5, which in turn makes the particles more stable. Using a slightly higher pH of 5.5 is also possible. A pH of 5.5 may decrease detrimental effects of acetate buffer on cell viability.

Additionally, using a high concentration of RNA in the RNA solution means that the amount of RNA in particles increase, which decreases the amount of particle solution that has to be administered to a cell or organism.

In another embodiment, the chitosan concentration is less than 250 μg/ml.

In a particular preferred embodiment, the chitosan concentration is less than 250 μg/ml, while the RNA concentration is higher than 100 μM.

By controlling the concentrations of RNA and chitosan, and thereby the N:P ratio, also the size of the particles can be controlled, (as documented in the examples section. Thus, in one embodiment, the size of the particle is between 10 and 200 nM.

In another embodiment, the formed particles are discrete in form and have a polydispersity index lower than 0,4.

A second aspect of the invention is a method of treating radiation induced fibrosis comprising administering an effective amount a chitosan-nucleic acid nanoparticle as in the first aspect to a person in need thereof. Specific embodiments of this aspect will be clear from the first aspect of the invention.

A second aspect of the invention is a method of treatment comprising administering an effective amount of a TNF-alpha antagonist to a subject in need thereof. Specific embodiments will be apparent from the first aspect.

The invention will now be described in further details in the following non-limiting examples.

### EXAMPLES

### Materials and Methods

Chemicals and siRNA

Chitosan (˜100 kDa, 84% deacetylation). TNF-α specific and control siRNA duplex was supplied by Integrated DNA Technologies, Inc. (Coralville, USA): TNF-α Dicer substrate (D-siRNAs) containing the sequence: sense, 5′-GUCUCAGCCUCUUCUCAUUCCUGCT-3′, antisense 3′-AGCAGGAAUGAGAAGGGCUGAGACAU-5′; D-siRNAs negative control containing the sequence: sense, 5′ CUUCCUCUCUUUCUCUCCCUUGUGA-3′, antisense 3′-UCACAAGGGAGAGAAAGAGAGGAAGGA-5′; siRNA negative control containing the sequence: sense, 5′-CGUUAAUCGCGUAUAAUACGCGUAT-3, antisense 3′-AUACGCGUAUUAUACGCGAUUAACGAC-5′

### Formation of Chitosan/siRNA Nanoparticles

Chitosan was dissolved in sodium acetate buffer (0.2M NaAc, pH 4.5) to obtain a 1 mg/ml solution and then adjusted to pH 5.5. 20 μl of siRNA (100 μm) in nuclease free water was added to 1 ml of filtered chitosan (1000 μg/ml) whilst stirring and left for 1 h. To calculate specific N:P ratios (defined as the molar ratio of chitosan amino groups/RNA phosphate groups) a mass-per-phosphate of 325 Da was used for RNA and mass-per-charge of chitosan 167.88 (84% deacetylation). Nanoparticles formed at ˜N:P 63

### The Leg Contracture Model was Used to Evaluate the Anti-Inflammatory Potential of Chitosan/siRNA Formulations.

Male CDF1 mice were divided into 9 groups of 3. Except of the control group with no treatment, all mice received a single irradiation dose of 45 Gy. Mice were i.p. dosed with 200 μl of chitosan/siRNA nanoparticles (5 μg TNF-α siRNA and 5 μg negative control) either 2 days before irradiation or 1 day after irradiation. The chitosan/siRNA nanoparticles treatment was continued twice a week and terminated on days 10, 22, 34, 225, and 258. One group of mice administered i.p. with sodium acetate buffer were used as a control.

The irradiated hind leg of the mice was scored for clinical symptoms of radiation-induced fibrosis by using the leg contracture model. Animals were assessed for clinical symptoms from scoring started at day 37 after irradiation and the last data from day 258.

Mice were terminated after the final scoring on day 258 except those showing severe signs of fibrosis which were sacrificied earlier.

Hind legs (irradiated and non-irradiated) and parts of liver, spleen, lung and kidney (one mouse from each group) were then frozen for following studies. Additionally, whole organs (liver, spleen, lung and kidney) were fixed in formalin for further histopathology studies (PIPELINE).

Knockdown of TNF-α was confirmed by using immunohistochemistry (localization of TNF-α in leg sections by the use of labeled TNF-α antibodies).

### Leg Contracture Model in Mice-Assay to Measure the Late Effect of Radiation

Leg contracture is defined as the difference in extensibility of the control and irradiated hind leg of mice.

### Measurement:

1) Acute score: phenotype of the irradiated
Acute score system:
   0.5=light reddening of the irradiated skin, <25% hair loss
   1.0=heavy reddening of the irradiated skin, 25-75% hair loss, edema
   1.5=aqueous reaction of a small skin area, 2 toes partly sticking together, >75% hair loss
   2.0=aqueous reaction of 25% of skin area, 2 toes sticking together but foot looks normal, all toes are identifiable
   2.5=aqueous reaction of 50% of skin area, 2 toes sticking together and form of the foot has changed, at least 3 toes are identifiable
   3.0=aqueous reaction of 75% of skin area, form of the foot is abnormal, 1 or 2 toes are identifiable
   3.5=aqueous reaction of whole skin area, abnormal form of the foot, no toes identifiable

2) Late score: stiffness of the leg
=> grade 0 control
=> grade 1-2 affected but not severe
=> grade 3-4 severe fibrosis

The acute score system represents the acute phase starting around day 10 after a radiation dose of 45 Gy. After 42 days the acute phase lapses and the late response on irradiation=RIF appears. At this time point we are using the late score system which is represented by the leg contracture model in mice.

### Example 1

### Radiation-Induced Fibrosis (RIF)

One of the most common long-term adverse effects is radiation-induced fibrosis (RIF), a complex biological process developing gradually over a number of years. RIF is a major problem in connection with radiation therapy in humans and is usually the limiting factor in the radiation dose given in connection with cancer therapy. It would therefore be desirable to develop a siRNA therapy that could be given in connection with radiation therapy. Thereby a higher or more frequent radiation dose could be administered with a presumed better effect on tumor growth. RIF is believed to occur as the result of a coordinated response to radiation involving several different cytokines and growth factors, fibroblast proliferation and differentiation, and also remodelling of the extra cellular matrix (ECM). A key player is TNF-α and it is suggested that downregulation of TNF-a expression in macrophages may have an overall beneficial effect on RIF development. To test this we set up a pilot experiment where groups of 3 CDF1 male mice where treated according to following scheme:

1. Control (no treatment)
2. Control+buffer 2d before irradiation until day 22
3. Control+siRNA mismatch 2d before irradiation until day 22
4a. siRNA TNFα 2d before irradiation until day 10
4b. siRNA TNFα 2d before irradiation until day 22
4c. siRNA TNFα 2d before irradiation until day 34
4d. siRNA TNFα 2d before irradiation until day 258
5a. siRNA TNFα 1d after irradiation until day 22
5b. siRNA TNFα 1d after irradiation until day 258

All groups except control group 1 received a radiation dose of 45 Gy at the behind-leg that previously have been shown to induce RIF after approximately 40 days (unpublished results). siRNA treatment was initiated as indicated and continued twice a week with a dose of 0.4 nmol siRNA in 200 microlitre siRNA. The induction of RIF is measured by a leg extension assay as described previously. The fibrotic tissue induces stiffness in the leg that can be measured by the distance the leg can be pulled under a constant force. The severity of the RIF is graded on a scale of 4, where 0 is equal to no symptoms and 4 is most server RIF.

### Conclusions

### Toxicity:

All mice 27 mice except one in group 5b survived the treatment. The death of this mouse was not investigated further but appeared not to be related to the treatment nor RIF. The data therefore suggest that the treatment is relatively non-toxic even after 18 doses of siRNA/chitosan.

### Efficacy in Inhibiting RIF Development:

No RIF symptoms were observed in any mice in groups that had received treatment with TNFa specific siRNA for 24 days or more Groups 4b, 4c, 4d, 3a and 5a). Group 4a, which received siRNA treatment for only 12 days did develop RIF suggesting that it is important that the treatment is prolonged. (at least until day 22 like group 4b).

No difference was observed dependent on whether the treatment was initiated 2 days before of 1 day after radiation (compare groups 4b and 5a and 4d and 5b).

All mice treated at least until day 22 or longer did not get fibrosis (groups 4b, 4c, 4d, 5a and 5b) whereas the control groups (1, 2 and 3) and group 4a (treated with TNFalpha siRNA only until day 10) developed fibrosis.

A novel treatment for arthritis using polymeric nanoparticles, based on systemic knockdown of TNF-α production for reduction in inflammation at local sites by i.p. targeting macrophages, has previously been demonstrated.

The present inventors have hypothesized that delivery of chitosan/siRNA nanoparticles targeting TNF-α may prevent radiation-induced fibrosis. Self-assembly driven nanoparticles formed between siRNA TNF-α or mismatch control and chitosan were used for in vivo silencing studies.

6 groups of 3 male CDF1 mice were dosed twice a week with 200 μl siRNA (100 μM) targeting TNF-α to investigate if a knockdown of TNF-α may prevent the development of fibrosis. Another group of mice was dosed with mismatch siRNA to confirm specific TNF-α silencing effects of the siRNA. Additionally, a group of 3 mice administered i.p. with sodium acetate buffer and an untreated group (no irradiation, no i.p. administration) were used as a control.

The therapeutic potential of silencing TNF-α production with chitosan/siRNA nanoparticles was investigated by using the leg contracture model in mice-an assay to measure the late effect of radiation. Leg contracture is thereby defined as the difference in extensibility of the control and irradiated hind leg of mice.

Differences in severe fibrosis within the different animal groups are derived from a fibrotic scoring system in FIG. 2 measuring the stiffness of the irradiated hind leg (scale: 0=normal, 1-2=mild to moderate fibrosis, 3-4=severe fibrosis).

All mice treated with chitosan/siRNA nanoparticles targeting TNF-α at least until day 22 did not develop fibrosis whereas mice treated with sodium acetate buffer or mismatch siRNA developed severe fibrosis (FIG. 2). Interestingly, mice treated with chitosan/siRNA nanoparticles targeting TNF-α until day 10 developed fibrosis. We conclude administration of chitosan/siRNA nanoparticles should be continued at least until day 22 and should be not terminated before the acute phase increases (data not shown).

### Example 2

Male CDF1 mice were divided into 16 groups of 6. Each group received irradiation in a different dose (40 Gy, 45 Gy, 50 Gy, 55 Gy, 60 Gy and 65 Gy). Altogether, 6 groups receiving irradiation dose as announced before were i.p. dosed with 200 μl of chitosan/siRNA nanoparticles (5 μg TNF-α), 6 groups received only irradiation, 2 groups receiving 50 and 55 Gy were i.p. dosed with 200 μl of chitosan/siRNA nanoparticles (5 μg negative control) and the last 2 groups receiving the same irradiation dose as the negative control group were i.p. dosed with sodium acetate buffer.

First, tumour cells were injected in 11 weeks old male CDF1 mice and chitosan/siRNA treatment was initiated as the tumour achieved a size of 200 mm3. The irradiation dose was given once 2 days after the first i.p. injection and the chitosan/siRNA treatment was continued twice a week for 3 month.

No effect of chitosan/siRNA treatment on tumour control was detected (FIG. 3).

Male CDF1 mice were divided into 4 groups of 8. Except of a control group with no treatment, all mice were i.p. dosed with 200 μl of chitosan/siRNA nanoparticles (5 μg TNF-α and 5 μg negative control) 2 days before tumour cell injection. Afterwards, the chitosan/siRNA treatment was continued twice a week for 3 month. Tumour growth was measured every day.

T-tests analysis showed no significant difference in tumour growth between control and siRNA groups (nor any other group comparisons) (FIG. 4).

### Example 3

### Chitosan/DsiRNA Nanoparticles have No Cytotoxic Side-Effects after Long-Term Treatment

To evaluate any possible systemic cytotoxic effect of long-term administration of chitosan/DsiRNA nanoparticle, mice that had been continuously bi-weekly treated were sacrificed after 258 days (total 78 i.p. injections) and liver, lung, spleen and kidney taken for histopathological analysis. No significant histological abnormalities were observed in the analysed organs between non-treated and TNFα DsiRNA treated animals (FIG. 5).

### Materials & Methods

### Cytotoxicity Analysis

Samples of the left and right kidney, liver, lung and spleen from the TNFα DsiRNA treated group (258 days) and the non-treated group were taken after termination of the study and preserved in formalin. Tissue samples were trimmed, processed, embedded in paraffin wax and sectioned at a nominal thickness of about 4 μm. All sections were stained with haematoxylin and eosin. At least one section of each organ sample was examined by undersigned pathologist Dr. Ortwin Vogel (Toxicologic Pathology Consultancy, Kiel, Germany) by light microscope.

### REFERENCES

Howard K A, Paludan S R, Behlke M A, Besenbacher F, Deleuran B, Kjems J. (2009). Chitosan/siRNA Nanoparticle-mediated TNF-α Knockdown in Peritoneal Macrophages for Anti-inflammatory Treatment in a Murine Arthritis Model. Mol Ther. 17: 162-168.
Distler J H, Schett G, Gay S, Distler O. (2008). The controversial role of tumor necrosis factor alpha in fibrotic diseases. Arthritis Rheum. 58:2228-35.

1. A method of treating or preventing radiation induced fibrosis, comprising:
providing a TNF-alpha antagonist to a subject in need thereof; and
evaluating fibrosis in said subject after providing said TNF-alpha antagonist.

2-10. (canceled)

11. The method of claim 1, wherein the TNF-alpha antagonist interacts with the mRNA encoding TNF-alpha or binds directly to TNF-alpha and said TNF-alpha antagonist is selected from the group consisting of an siRNA specific for an mRNA encoding TNF-alpha, an antibody, an aptamer, a soluble TNF-alpha receptor, or an RNase H inducing antisense oligonucleotide specific for an mRNA encoding TNF-alpha, and a ribozyme specific for an mRNA encoding TNF-alpha.

12. The method of claim 1, wherein said TNF-alpha antagonist is an siRNA.

13. The method of claim 12, wherein said siRNA is present in a chitosan/siRNA nanoparticle.

14. The method of claim 11, wherein said TNF-alpha antagonist is formulated for intraperitoneal administration.

15. The method of claim 1, wherein said TNF-alpha antagonist is provided prior to initiation of radiation therapy.

16. The method of claim 16, wherein said TNF-alpha antagonist is provided at least 24 hours before radiation therapy.

17. The method of claim 16, wherein said TNF-alpha antagonist is provided no more than 1 week before radiation therapy.

18. The method of claim 15, wherein repeating cycles of administration of said TNF-alpha antagonist and radiation therapy is performed.

19. A method of treating or preventing radiation induced fibrosis, comprising:
providing a TNF-alpha antagonist to a subject in need thereof, wherein said TNF-alpha antagonist is a chitosan/siRNA nanoparticle and, wherein said siRNA is specific for an mRNA encoding TNF-alpha; and
evaluating fibrosis in said subject after providing said TNF-alpha antagonist.


SEQUENCE LISTING (6 sequences)

SEQ ID NO: 1 - DNA, 25 bp, Artificial Sequence
  siRNA oligonucleotide
gucucagccucuucucauuccugct

SEQ ID NO: 2 - DNA, 26 bp, Artificial Sequence
  siRNA oligonucleotide
uacagagucgggaagaguaaggacga

SEQ ID NO: 3 - DNA, 25 bp, Artificial Sequence
  siRNA oligonucleotide
cuuccucucuuucucucccuuguga

SEQ ID NO: 4 - DNA, 27 bp, Artificial Sequence
  siRNA oligonucleotide
aggaaggagagaaagagagggaacact

SEQ ID NO: 5 - DNA, 25 bp, Artificial Sequence
  siRNA oligonucleotide
cguuaaucgcguauaauacgcguat

SEQ ID NO: 6 - DNA, 27 bp, Artificial Sequence
  siRNA oligonucleotide
cagcaauuagcgcauauuaugcgcaua